(19) United States
(12) Patent Application Publication
(10) Pub. No.: US 2010/0114309 A1
(43) Pub. Date: May 6, 2010

(54) DRUG DELIVERY IMPLANTS FOR INHIBITION OF OPTICAL DEFECTS

(76) Inventors: Eugene de Juan, JR., San Francisco, CA (US); Cary J. Reich, Los Gatos (CA); Stephen Boyd, Murrieta (CA); Hanson S. Gifford, Woodside, CA (US); Mark Deem, Mountain, CA (US)

Correspondence Address: SCHWEGMAN, LUNDBERG & WOESSNER, P.A., P.O. BOX 2938, MINNEAPOLIS, MN, 55402, US

(21) Appl. No.: 12/521,543

(22) PCT Filed: Dec. 21, 2007
(86) PCT No.: PCT/US07/88701

§ 371 Date: Dec. 31, 2009

Related U.S. Application Data
(60) Provisional application No. 60/871,867, filed on Dec. 26, 2006.

(51) Int. Cl. A61F 2/16

(52) U.S. Cl. 623/6.39; 623/6.62

(57) ABSTRACT

An implant for use with an eye comprises an implantable structure and a therapeutic agent. The therapeutic agent is deliverable from the structure into the eye so as to therapeutically effect and/or stabilize a refractive property of the eye. In many embodiments, the refractive property of the eye may comprise at least one of myopia, hyperopia or astigmatism. The therapeutic agent can comprise a composition that therapeutically effects or stabilizes the refractive property of the eye. The therapeutic agent may comprise at least one of a mydriatic or a cycloplegic drug. For example, the therapeutic agent may include a cycloplegic that comprises at least one of atropine, cyclopentolate, succinylcholine, homatropine, scopolamine, or tropicamide. In many embodiments, a retention element can be attached to the structure to retain the structure along a natural tissue surface.

### CROSS-REFERENCES TO RELATED APPLICATIONS

This application claims the benefit of under 35 U.S.C. §109(e) of U.S. Provisional Patent Application No. 60/871,867 filed on Dec. 26, 2007, the disclosure of which is incorporated herein by reference.

### BACKGROUND OF THE INVENTION

The present invention is directed to the treatment of optical defects of the eye with implants that release one or more therapeutic agents.

Pathological conditions that degrade vision can be debilitating. Optical defects of the eye that interfere with one's ability to see can range in severity from nearly imperceptible to blindness. One common form of optical defect of the eye is refractive error of the eye, with typical refractive errors including nearsightedness or myopia, farsightedness or hyperopia, and astigmatism. Refractive error of the eye generally results from imperfection in the physical properties of the ocular tissues of the eye so that an image formed on the retina is less than ideal. The eye includes an anterior corneal surface and intermediate crystalline lens, both of which refract light to form an image on the retina. Imperfections in either the cornea or the crystalline lens can result in refractive error of the eye. The positions of the cornea and crystalline lens in relation to each other and in relation to the retina can also effect image quality and refractive error. For example, if the distance from the crystalline lens to the retina is too long, a patient can suffer from myopia. Current eye research and treatments are also directed to the diagnosis and correction of additional refractive errors of the eye such as spherical aberration and coma.

Refractive errors of the eye can be corrected by treatments that include eye glasses, intraocular lenses, contact lenses and laser surgery. Although these treatments are generally effective, each treatment modality has limitations and may not be suitable for everyone. For example, eyeglasses and contact lenses are not a permanent form of correction and are only effective while worn. Thus, many people suffer from significant degradation in their vision when these lenses are not worn. Intraocular lenses are invasive and require surgery, so that the use of intraocular lenses is often limited to the treatment of cataracts. Although laser eye surgery is effective this elective surgery can occasionally result in complications, so that many people choose to live the inconvenience and limitations of eyeglasses and/or contact lenses. In addition to the above limitations, these therapies generally attempt to correct optical defects of an eye after the defect has developed.

There have been proposals to control the progression of refractive error. For example, the application of atropine eye drops to children has been shown to control the progression of myopia. However, the application of liquid drops with atropine can result in side effects and may involve applying liquid drops regularly for an extended time. In addition, the eye drop format can be difficult to instill in children making compliance a significant issue in treatment. As such, since compliance to the drop regimen may be determinative to the desired clinical outcome, missing doses can lead to further disease progression.

In light of the above, what is needed are treatments for optical defects of the eye that eliminate at least some of the above short comings of the current therapies.

### BRIEF SUMMARY OF THE INVENTION

The present invention is directed to the treatment of optical defects of the eye with implants that release a therapeutic agent.

In a first aspect, the present invention provides an implant for use with an eye. The implant comprises an implantable structure and a therapeutic agent. The therapeutic agent is deliverable from the structure into the eye so as to therapeutically effect and/or stabilize a refractive property of the eye.

In many embodiments, the refractive property of the eye may comprise at least one of myopia, hyperopia or astigmatism. The therapeutic agent can comprise a composition that therapeutically effects or stabilizes the refractive property of the eye when delivered into at least one of a sclera, a vitreous humor, an aqueous humor or a ciliary muscle of the eye. The therapeutic agent may comprise at least one of a mydriatic or a cycloplegic drug. For example, the therapeutic agent may include a cycloplegic that comprises at least one of atropine, cyclopentolate, succinylcholine, homatropine, scopolamine, or tropicamide.

In many embodiments, a retention element can be attached to the structure to retain the structure along a natural tissue surface of or adjacent to the eye. The retention element can be shaped to retain the structure in or adjacent at least one of a punctual duct, a scleral tissue, or a conjunctival tissue. The structure can be shaped to retain the structure adjacent at least one of a punctual duct, a scleral tissue, or a conjunctival tissue. The structure may have at least one surface and release a therapeutic quantity of the therapeutic agent into tear or tear film fluid of the eye throughout a time period of at least one week when the implant is implanted with the at least one surface exposed to the tear or tear film fluid. For example, the structure can be adapted to release the therapeutic agent in therapeutic amounts over a period of time from about one to twelve months after the structure is inserted into the eye, and the structure may comprise at least one of a reservoir, a matrix, a solution, a surface coating or a bioerodable material. The structure may comprise a drug core and a layer disposed over the drug core to inhibit release of the therapeutic agent through the layer, and the layer may comprise an opening formed therein to release the drug through the opening. The structure may comprise particles of the agent, and the particles may independently release the agent therefrom when the structure is implanted to provide a substantially uniform release rate.

In specific embodiments, at least a portion of the structure may be bioerodable, and the therapeutic agent can be released while the structure erodes.

Many embodiments may comprise a counteractive agent to avoid a side effect of the therapeutic agent, and the counteractive agent may comprise at least one of an anti-glaucoma drug or a miotic drug. For example, the anti-glaucoma drug may comprise at least one of a sympathomimetic, a parasympathomimetic, a beta blocking agent, a carbonic anhydrase inhibitor, or prostaglandin analogue. In specific embodiments, the anti-glaucoma drug may comprise at least one of Apraclonidine, Brimonidine, Clonidine, Dipivefrine, Epinephrine, Aceclidine, Acetylcholine, Carbachol, Demecarium, Echothiophate, Fluostigmine, Neostigmine, Paraoxon, Physostigmine, Pilocarpine, Acetazolamide, Brinzolamide, Diclofenamide, Dorzolamide, Methazolamide, Befunolol, Betaxolol, Carteolol, Levobunolol, Metipranolol, Timolol, Bimatoprost, Latanoprost, Travoprost, Unoprostone, Dapiprazole or Guanethidine.

In specific embodiments, a therapeutic implant comprises a structure, a punctal plug and a therapeutic agent. The punctual plug retains the structure adjacent to an eye. The therapeutic agent may comprises atropine deliverable from the structure into the eye to therapeutically effect and/or stabilize refractive properties of the eye. The refractive property of the eye may comprise at least one of myopia, astigmatism or hyperopia.

In another aspect a method of treating an optical defect of an eye with a therapeutic agent is provided. The method comprises implanting a structure into a tissue of or near the eye. A therapeutic agent is released from the implanted structure so that the therapeutic agent effects and/or stabilizes a refractive property of the eye.

In some embodiments, the refractive property of the eye comprises at least one of a myopia, a hyperopia or an astigmatism. The therapeutic agent can be released in therapeutic amounts over a period of time from about one to twelve months after the structure is inserted into the eye. For example, the period of time can be from about six to twelve months. The therapeutic agent can be continuously released over the period of time.

In many embodiments, the structure can be implanted in at least one of a sclera, a punctum or a conjunctiva of the eye. For example, the structure may be anchored to the punctum and release the therapeutic agent into a tear or tear film of the eye. In addition or in combination, the structure may be anchored to the sclera and release the therapeutic agent into at least one of a vitreous humor, an aqueous humor or a ciliary muscle of the eye. The structure may be anchored to the conjunctiva and release the therapeutic agent into at least one of a vitreous humor, an aqueous humor or a ciliary muscle of the eye. The structure may be covered by the conjunctiva and release the therapeutic agent into at least one of a vitreous humor, an aqueous humor or a ciliary muscle of the eye. For example, the structure is placed between the conjunctiva and the sclera.

In many embodiments, the therapeutic agent effects accommodation of the eye. In specific embodiments, the therapeutic agent can comprise a cycloplegic, such as at least one of atropine, cyclopentolate, succinylcholine, homatropine, scopolamine, or tropicamide. The therapeutic agent can comprise atropine.

In some embodiments a counteractive agent can be released from the implanted structure and/or another structure to counteract a side effect of the therapeutic agent. The counteractive agent may comprise at least one of an anti-glaucoma drug or a miotic drug. In specific embodiments, the anti-glaucoma drug may comprise at least one of a sympathomimetic, a parasympathomimetic, a beta blocking agent, a carbonic anhydrase inhibitor, or prostaglandin analogue.

In some embodiments the therapeutic agent can be released with a profile that corresponds to a kinetic order of therapeutic agent release and the order can be within a range from about zero to about one. In specific embodiments, the range is from about zero to about one half, for example from about zero to about one quarter. The therapeutic agent may released with a profile that corresponds to a kinetic order of therapeutic agent release and the order is within a range from about zero to about one half for at least about a month after the structure is inserted, for example the order can be within the range at least about 3 months after the structure is inserted.

In some embodiments, a method of treating an optical defect of an eye comprises treating the eye with at least one of an anti-glaucoma drug and/or a miotic drug to avoid a side effect of a therapeutic agent used to treat the optical defect of the eye. Children and/or adolescents may treated, and the optical defect of the eye may comprise at least one of a myopia, a hyperopia or an astigmatism. The anti-glaucoma drug may comprise at least one of a sympathomimetic, a parasympathomimetic, a beta blocking agent, a carbonic anhydrase inhibitor, or prostaglandin analogue. In specific embodiments, the anti-glaucoma drug comprises at least one of Apraclonidine, Brimonidine, Clonidine, Dipivefrine, Epinephrine, Aceclidine, Acetylcholine, Carbachol, Demecarium, Echothiophate, Fluostigmine, Neostigmine, Paraoxon, Physostigmine, Pilocarpine, Acetazolamide, Brinzolamide, Diclofenamide, Dorzolamide, Methazolamide, Befunolol, Betaxolol, Carteolol, Levobunolol, Metipranolol, Timolol, Bimatoprost, Latanoprost, Travoprost, Unoprostone, Dapiprazole or Guanethidine. In many embodiments, the anti-glaucoma drug is capable of a miotic effect. The miotic drug can comprise at least one of echothiophate, pilocarpine, physostigmine salicylate, diisopropylfluorophosphate, carbachol, methacholine, bethanechol, epinephrine, dipivefrin, neostigmine, echothiopateiodide or demecium bromide.

### BRIEF DESCRIPTION OF THE DRAWINGS

FIGS. 1-1 and 1-2 show anatomical tissue structures of the eye suitable for use with implants, according to embodiments of the present invention;

FIG. 1A shows a top cross sectional view of a sustained release implant to treat an optical defect of an eye, according to an embodiment of the present invention;

FIG. 1B shows a side cross sectional view of the sustained release implant of FIG. 1A;

FIG. 1C shows a perspective view of a sustained release implant with a coil retention element, according to an embodiment of the present invention;

FIG. 1D shows a perspective view of a sustained release implant with a retention element comprising struts, according to an embodiment of the present invention;

FIG. 1E shows a perspective view of a sustained release implant with a cage retention element, according to an embodiment of the present invention;

FIG. 1F shows a perspective view of a sustained release implant comprising a core and sheath, according to an embodiment of the present invention;

FIG. 2A shows a cross sectional view of a sustained release implant with core comprising an enlarged exposed surface area, according to an embodiment of the present invention;

FIG. 2B shows a cross sectional view of a sustained release implant with a core comprising an enlarged exposed surface area, according to an embodiment of the present invention;

FIGS. 2C and 2D show perspective view and cross sectional views, respectively, of a sustained release implant with a core comprising a reduced exposed surface area, according to an embodiment of the present invention;

FIG. 2E shows a cross sectional view of a sustained release implant with a core comprising an enlarged exposed surface area with castellation, according to an embodiment of the present invention;

FIG. 2F shows a perspective view of a sustained release implant comprising a core with redundant surface area according to an embodiment of the present invention;

FIG. 2G shows a perspective view of a sustained release implant with a core comprising a channel with an internal porous surface, according to an embodiment of the present invention;

FIG. 2H shows a perspective view of a sustained release implant with a core comprising porous channels to increase drug migration, according to an embodiment of the invention;

FIG. 2I shows a perspective view of a sustained release implant with a convex exposed drug core surface, according to an embodiment of the present invention;

FIG. 2J shows a side view of a sustained release implant with a core comprising an exposed surface area with several soft protrusions, tendrils, cilia type members extending therefrom, according to an embodiment of the present invention;

FIG. 2K shows a side view of a sustained release implant with a drug core comprising a convex exposed surface and a retention element, according to an embodiment of the present invention.

FIG. 3A shows a perspective view of a punctual plug with a reservoir, according to an embodiment of the present invention;

FIG. 3B shows a schematic representation of a preferred configuration of medication within the reservoir and its contact with the external tear flow, according to an embodiment of the present invention;

FIG. 4 shows a retention element that encompass a tube, for example a tube used to form a punctual plug, and a structure to release therapeutic agents that encompass a drug reservoir enclosed with a permeable layer, according to an embodiment of the present invention;

FIG. 5 show a retention elements that encompasses a punctual plug, and a structure to release therapeutic agents that encompasses a drug reservoir enclosed with a material permeable to the drug, according to an embodiment of the present invention;

FIG. 6 shows a punctual plug having materials to release therapeutic agents (e.g. coatings and/or biodegradable polymers) according to embodiments of the present invention;

FIG. 7 shown an implant for complete insertion into the canaliculus of the human eye with medication, according to an embodiment of the present invention;

FIG. 8A shows a plan view, with representative dimensions, of a punctal plug according to an embodiment of the present invention;

FIG. 8B shows a plan view, with representative dimensions, of a punctal plug, according to an embodiment of the present invention;

FIG. 9 shows a retention element that encompasses a punctual plug and a retention element that encompasses a hollow implant, and structures to release therapeutic agents that encompass coatings applied to the retention elements, according to an embodiment of the present invention; and

FIGS. 10A to 10C show deployment of a sustained release implant, according to an embodiment of the present invention;

FIG. 11 shows sustained release therapeutic agent implants and implant locations on or near an eye, according to embodiments of the present invention;

FIG. 12A shows a device for treating optical defects of the eye that comprises a sustained release implant that releases a therapeutic agent to treat the optical defect of the eye and additional sustained release implants to counteract side effects of the therapeutic agent; and

FIG. 12B shows a sustained release implant that releases a therapeutic agent to treat an optical defect of the eye and releases counteractive agents that counteracts a side effect of the therapeutic agent, according to embodiments of the present invention.

### DETAILED DESCRIPTION OF THE INVENTION

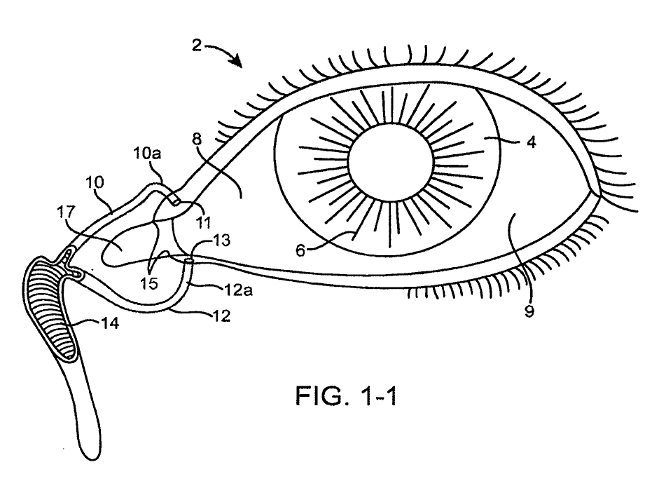

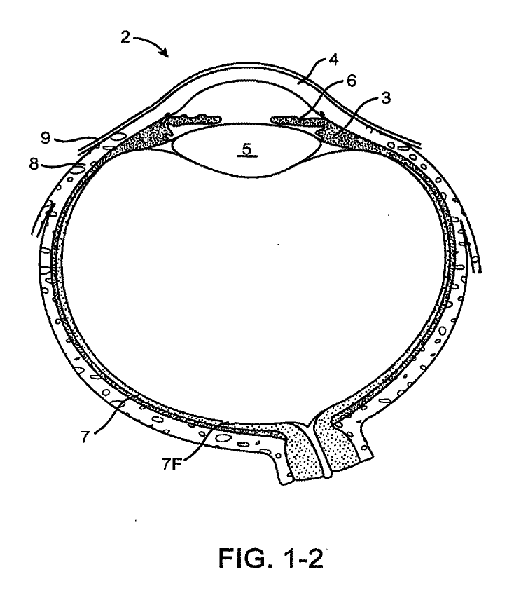

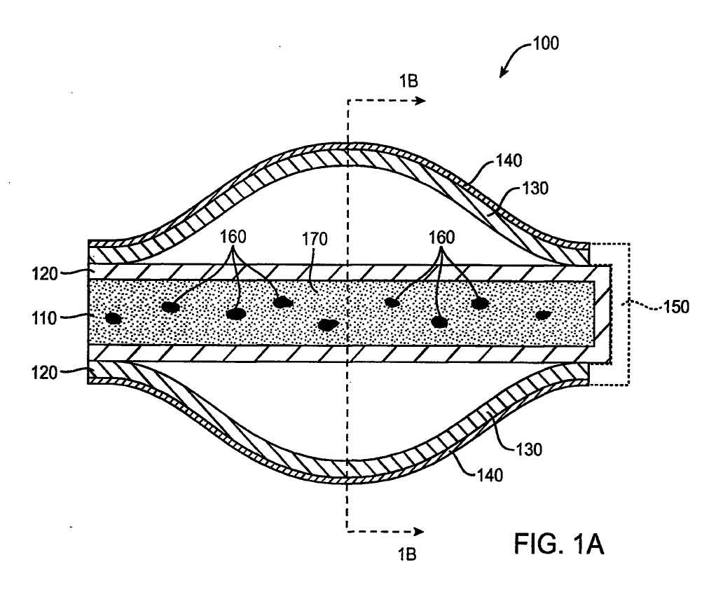

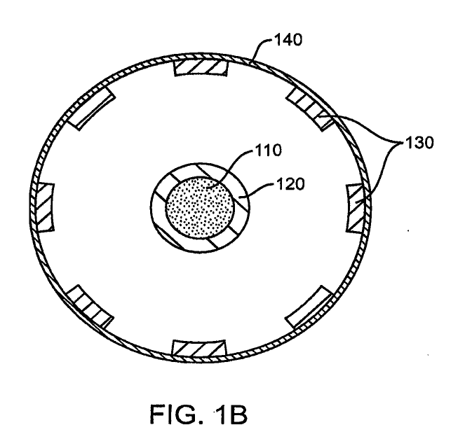

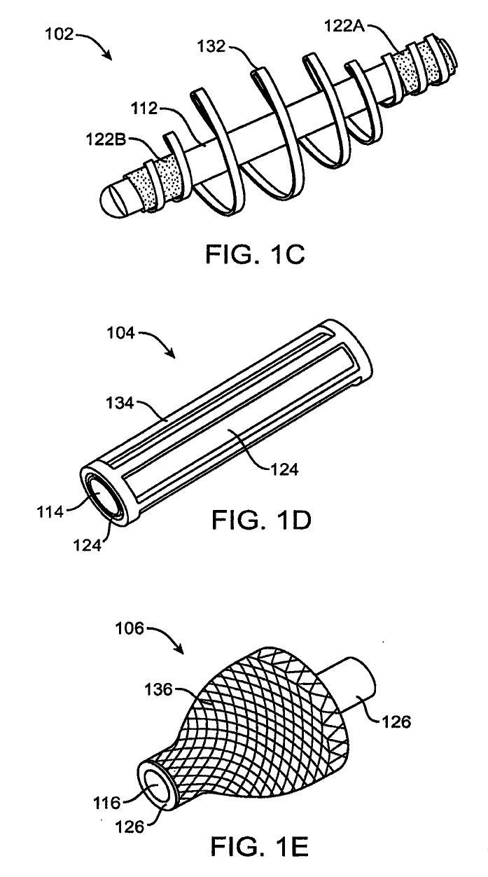

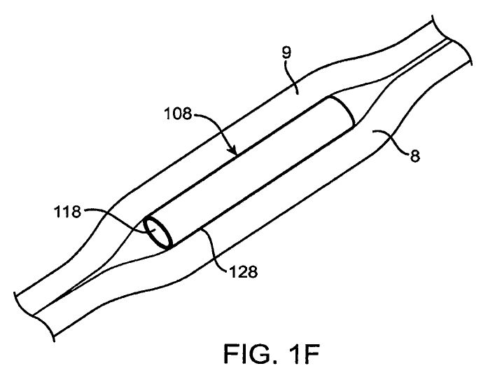

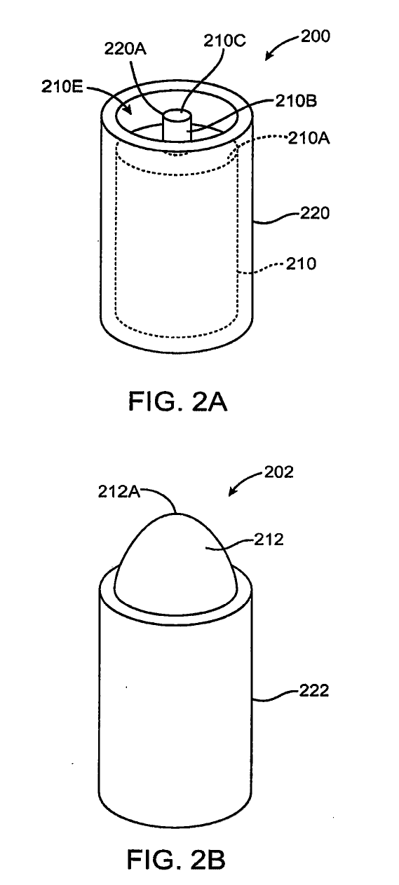

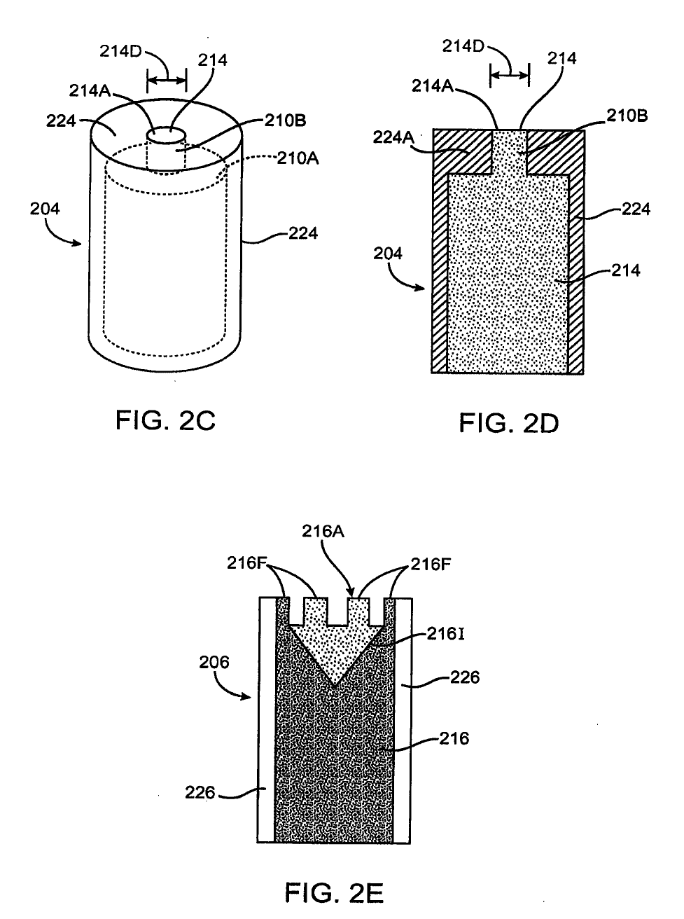

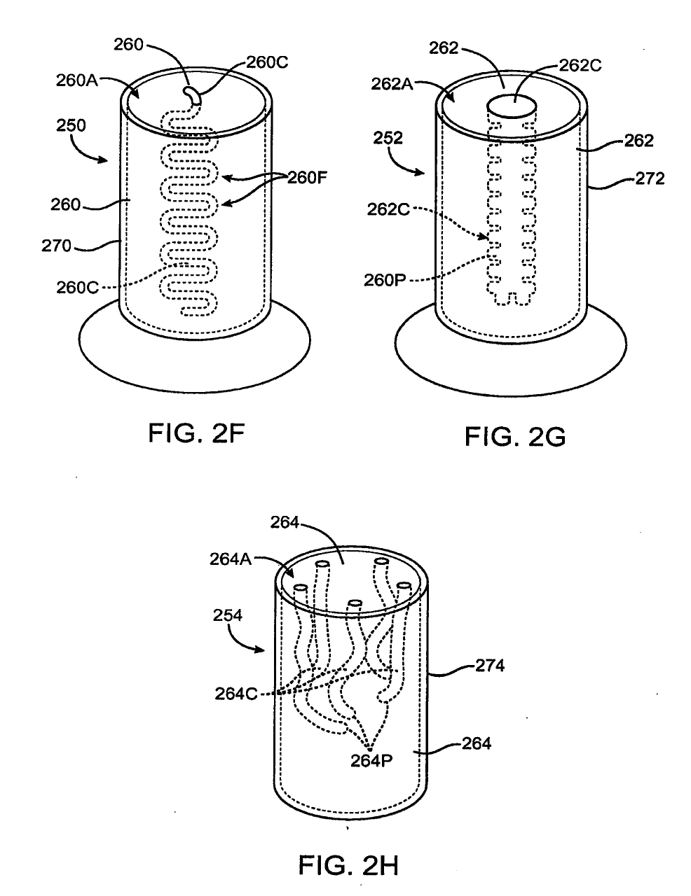

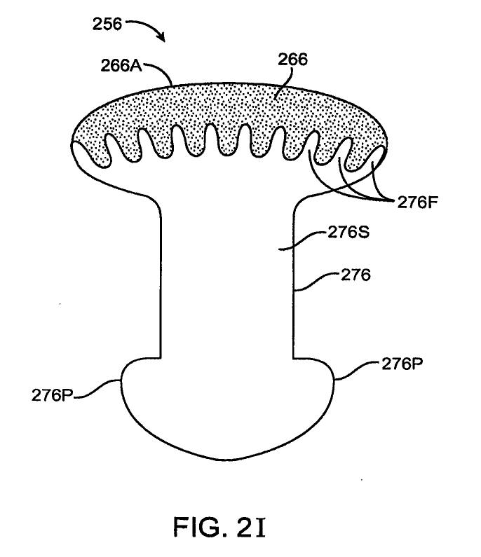

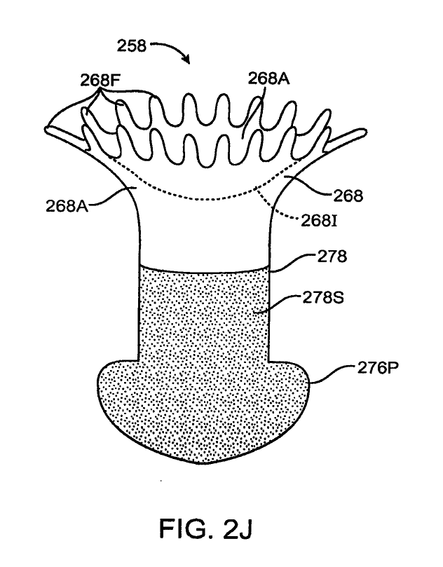

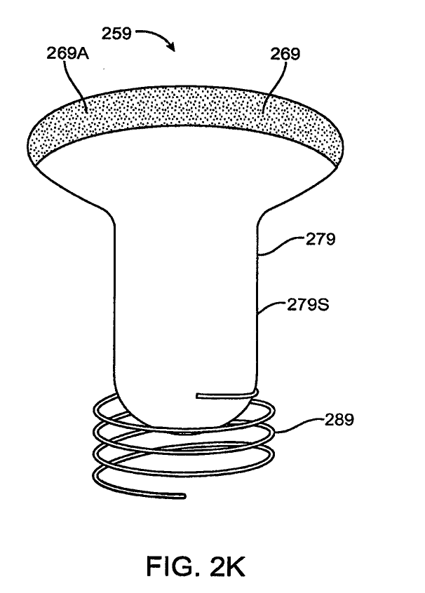

FIGS. 1-1 and 1-2 show anatomical tissue structures of an eye 2 suitable for treatment with implants, according to an embodiment of the present invention. Eye 2 includes a cornea 4 and an iris 6. A sclera 8 surrounds cornea 4 and iris 6 and appears white. A conjunctival layer 9 is substantially transparent and disposed over sclera 8. A crystalline lens 5 is located within the eye. A retina 7 is located near the back of eye 2 and is generally sensitive to light. Retina 7 includes a fovea 7F that provides high visual acuity and color vision. Cornea 4 and lens 5 refract light to form an image on fovea 7F and retina 7. The optical power of cornea 4 and lens 5 contribute to the formation of images on fovea 7F and retina 7. The relative locations of cornea 4, lens 5 and fovea 7F are also important to image quality. For example, if the axial length of eye 2 from cornea 4 to retina 7F is large, eye 2 can be myopic. Also, during accommodation, lens 5 moves toward cornea 4 to provide good near vision of objects proximal to the eye.

The anatomical tissue structures shown in FIG. 1-1 also include the lacrimal system, which includes an upper canaliculus 10 and a lower canaliculus 12, collectively the canaliculae, and the naso-lacrimal duct or sac 14. The upper and lower canaliculae terminate in an upper punctum 11 and a lower punctum 13, also referred to as punctal apertures. The punctal apertures are situated on a slight elevation at the medial end of the lid margin at the junction 15 of the ciliary and lacrimal portions near the medial canthus 17. The punctal apertures are round or slightly ovoid openings surrounded by a connective ring of tissue. Each of the punctal openings 11, 13 leads into a vertical portion 10a, 12a of the respective canaliculus before turning horizontally to join its other canaliculus at the entrance of a lacrimal sac 14. The canaliculae are tubular and lined by stratified squamous epithelium surrounded by elastic tissue which permits the canaliculus to be dilated.

As the eye is an optical system, the interrelationship of the optical components of the eye can contribute to a refractive defect of the eye (e.g. myopia, hyperopia and/or astigmatism). In some instances, if the eye attains an axial length that is too long, the eye can be myopic. Also, if the cornea and/or the lens have excessive optical power relative to the length of the eye, the eye may be myopic. If the cornea and/or lens have insufficient optical power relative to the width of the eye, hyperopia can occur (i.e. the axial length of the eye is too short relative to the width of the eye). The position of the crystalline lens within the eye may also contribute to the refractive condition of the eye as well.

Growth and development of the eye during childhood and adolescence can effect the optical properties of the eye, and many people undergo a progressive worsening of refractive error of the eye during childhood and adolescence. For example, myopic school age children can undergo a progressive worsening of myopia as the eye develops and grows. As this progression of myopia is associated with development of the eye during childhood and adolescence it can be referred to as developmental myopia. Also, as moderate to severe myopia can be associated with astigmatism, treatment of the progressive worsening of myopia can also treat the progressive worsening of astigmatism.

In preferred embodiments, the progression of a refractive defect of the eye is treated with a therapeutic agent to attenuate the worsening of the refractive defect. The therapeutic agent can be a cycloplegic, for example atropine, that is used to attenuate the progression of myopia. Although such treatments may not entirely eliminate refractive defects of the eye, early detection and intervention can limit the severity of the refractive defect.

FIG. 1A shows a top cross sectional view of a sustained release implant 100 to treat an optical defect of an eye, according to embodiments of the present invention. Implant 100 includes a drug core 110. Drug core 110 is an implantable structure that retains a therapeutic agent. Drug core 110 comprises a matrix 170 that contains particles 160 of therapeutic agent. Particles 160 will often comprise a concentrated form of the therapeutic agent, for example a solid form such as a crystalline form and/or liquid form such as an oil form of the therapeutic agent, and the therapeutic agent may over time dissolve into matrix 170 of drug core 110. Matrix 170 can comprise a silicone matrix or the like.

Drug core 110 is surrounded by a sheath body 120. Sheath body 120 is can be substantially impermeable to the therapeutic agent, so that the therapeutic agent is often released from an exposed surface on an end of drug core 110 that is not covered with sheath body 120. A retention element 130 is connected to drug core 110 and sheath body 120. Retention element 130 is shaped to retain the implant in a hollow tissue structure, for example, a punctum of a canaliculus as described above.

An occlusive element 140 is disposed on and around retention element 130. Occlusive element 140 is impermeable to tear flow and occludes the hollow tissue structure and may also serve to protect tissues of the tissue structure from retention element 130 by providing a more benign tissue-engaging surface. Sheath body 120 includes a sheath body portion 150 that connects to retention element 130 to retain sheath body 120 and drug core 110. Sheath body portion 150 also acts as a stop to limit movement of sheath body 120 and drug core 110.

FIG. 1B shows a side cross sectional view of the sustained release implant of FIG. 1A. Drug core 110 is cylindrical and shown with a circular cross-section. Sheath body 120 comprises an annular portion disposed on drug core 110. Retention element 130 comprises several longitudinal struts 131. Longitudinal struts 131 are connected together near the ends of the retention element. Although longitudinal struts are show, circumferential struts can also be used. Occlusive element 140 is supported by and disposed over longitudinal struts 131 of retention element 130 and may comprise a radially expandable membrane or the like.

The drug core comprises the therapeutic agent and materials to provide sustained release of the therapeutic agent. The therapeutic agent, for example atropine, migrates from the drug core to the target tissue, for example ciliary muscles of the eye. The therapeutic agent may optionally be only slightly soluble in the matrix so that the release rate remains “zero order” for the lifetime of the release of the therapeutic agent when dissolved in the matrix and available for release from the surface of drug core 110. As the therapeutic agent diffuses from the exposed surface of the core to the tear or tear film, the rate of migration from the core to the tear or tear film is related to the concentration of therapeutic agent dissolved in the matrix. In some embodiments, the concentration of therapeutic agent dissolved in the drug core may be controlled to provide the desired rate of release of the therapeutic agent. The therapeutic agent included in the core can include liquid, solid, solid gel, solid crystalline, solid amorphous, solid particulate, and/or dissolved forms of the therapeutic agent. In some embodiments, the drug core comprises a silicone matrix containing the therapeutic agent. An exemplary therapeutic agent comprises solid atropine particles dispersed in the silicone matrix.

The drug core can be made from any biocompatible material capable of providing a sustained release of the therapeutic agent. Although the drug core is described above with respect to an embodiment comprising a matrix with a substantially non-biodegradable silicone matrix with particles of the drug located therein that dissolve, the drug core can include any structure that provides sustained release of the therapeutic agent, for example biodegradable matrix, a porous drug core, liquid drug cores and solid drug cores. The structures can be adapted to release the therapeutic agent in therapeutic amounts over a period of time from about one to twelve months after the structure is inserted into the eye. A matrix that contains the therapeutic agent can be formed from either biodegradable or non-biodegradable polymers. Examples of biodegradable polymers may include poly(L-lactic acid) (PLLA), poly(L-glycolic acid) (PLGA), polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid), collagen matrices and combinations thereof. The devices of the present invention may be fully or partially biodegradable or non-biodegradable. Examples of non-biodegradable materials are various commercially available biocompatible polymers including but not limited to silicone, polyethylene terephthalate, acrylates, polyethylenes, polyolefins, including ultra high molecular weight polyethylene, expanded polytetrafloroethylene, polypropylene, polycarbonate urethane, polyurethanes, polyamides, sheathed collagen. In some embodiments the drug core may comprise a hydrogel polymer, either degradable or non-degradable. In some embodiments, the therapeutic agent can be comprised in a drug eluting material used as a coating, such as those commercially available from Surmodics of Eden Prairie, Minn., and Angiotech Pharmaceuticals of British Columbia, Canada, and the like.

The therapeutic agent can comprise any substance, for example a drug, that effects the optical properties of the eye. Suitable drugs to effect the optical properties of the eye may include cycloplegics, for example atropine, cyclopentolate, succinylcholine, homatropine, scopolamine, and/or tropicamide. Other drugs may be used to effect pupil dilation and/or other optical properties of the eye include neostigmine, phentolamine, phospholine iodide and pilocarpine. Additional drugs such as miotics can be used, including echothiophate, pilocarpine, physostigmine salicylate, diisopropylfluorophosphate, carbachol, methacholine, bethanechol, epinephrine, dipivefrin, neostigmine, echothiopateiodide and demecium bromide. Other suitable therapeutic agents include mydriatics such as hydroxyamphetamine, ephedrine, cocaine, tropicamide, phenylephrine, cyclopentolate, oxyphenonium and eucatropine. In addition, anti-cholinergics may be employed such as, pirenzepine. Examples of applicable therapeutic agents may be found in United States Patent Applications 20060188576 and 20030096831, hereby incorporated by reference in their entirety.

In addition to the therapeutic agent used to treat the optical defect of the eye, additional therapeutic agents can be provided to counteract possible side effects of the therapeutic agent. The additional counteractive therapeutic agent(s) can be comprised within the core that releases the therapeutic agent that treats the optical defect of the eye, or additional drug cores can be provided to separately release the additional counteractive therapeutic agent(s).

One possible side effect of a cycloplegic therapeutic agent is pupil dilation that can result in photophobia. Therefore, in some embodiments, a miotic therapeutic agent is released into the eye to counteract the pupil dilation caused by the cycloplegic.

Another potential side effect of cycloplegic therapeutic agents is glaucoma, possibly related to the dilation of the pupil. Therefore, in some embodiments an anti-glaucoma therapeutic agent(s) may be released to counteract a possible glaucoma inducing side effect of the therapeutic agent used to treat the optical defect of the eye. Suitable anti-glaucoma therapeutic agents include: sympathomimetics such as Apraclonidine, Brimonidine, Clonidine, Dipivefrine, and Epinephrine; parasympathomimetics such as Aceclidine, Acetylcholine, Carbachol, Demecarium, Echothiophate, Fluostigmine, Neostigmine, Paraoxon, Physostigmine, and Pilocarpine; carbonic anhydrase inhibitors such as Acetazolamide, Brinzolamide, Diclofenamide, Dorzolamide, and Methazolamide, beta blocking agents such as Befunolol, Betaxolol, Carteolol, Levobunolol, Metipranolol, and Timolol; prostaglandin analogues such as Bimatoprost, Latanoprost, Travoprost, and Unoprostone; and other agents such as Dapiprazole, and Guanethidine. In a preferred embodiment, atropine is released as a therapeutic agent to treat developmental myopia in children, and bimatoprost and/or latanoprost is released as an anti-glaucoma treatment.

It should be noted that some therapeutic agents will have more than one effect on the eye. For example, anti-glaucoma therapeutic agents can also cause pupil constriction. Thus in some embodiments, an additional therapeutic agent can be added to counteract more than one side effect of the therapeutic agent that is released to correct the optical defect of the eye.

The therapeutic agent is released at therapeutic levels to provide a desired treatment response when implant 100 is implanted in a tissue or near the eye. For example, with the drug atropine as used to treat myopia, the atropine is released from the drug core at therapeutic rate that delivers the lowest effective dose. The drug is preferably released at a uniform rate, for example a rate that corresponds to zero order kinetics, although the drug can be released at rates that correspond to other orders of reaction kinetics, for example first order. In many embodiments, the kinetic order of the reaction will vary from zero order to first order as the drug is released. Thus, the therapeutic agent is released with a profile that corresponds to a range of kinetic orders that varies from about zero to about one. Ideally, the drug core is removed before the rate at which the therapeutic agent is released changes significantly so as to provide uniform delivery of the therapeutic agent. As a uniform rate of delivery is desired, it may be desirable to remove and/or replace the drug core before the reaction kinetics transition entirely to first order. In other embodiments, first or higher order release kinetics may be desirable during some or all of the treatment, so long as the therapeutic agent release profile remains within a safe and effective range. In some embodiments the drug core may release at an effective rate for the period of 1 week to 5 years, more particularly in the range of 3-24 months.

The rate of release of the therapeutic agent can be related to the concentration of therapeutic agent dissolved in the drug core. In many embodiments, the drug core comprises non-therapeutic agents that are selected to provide a desired solubility of the therapeutic agent in the drug core. The non-therapeutic agent of the drug core can comprise polymers as described above and additives. A polymer of the core can be selected to provide the desired solubility of the therapeutic agent in the matrix. For example, the core can comprise hydrogel that may promote solubility of hydrophobic treatment agent. In some embodiments, functional groups can be added to the polymer to modulate the release kinetics of the therapeutic agent in the matrix. For example, functional groups can be attached to silicone polymer.

In some embodiments, additives may be used to control the concentration of therapeutic agent by increasing or decreasing solubility of the therapeutic agent in the drug core. The solubility may be controlled by providing appropriate molecules and/or substances that increase and/or decrease the solubility of the dissolved form of the therapeutic agent to the matrix. The solubility of the dissolved form of the therapeutic agent may be related to the hydrophobic and/or hydrophilic properties of the matrix and therapeutic agent. For example, surfactants, salts, hydrophilic polymers can be added to the matrix to modulate the release kinetics. In addition, oils and hydrophobic molecules can be added to the matrix to modulate the release kinetics of the matrix.

Instead or in addition to controlling the rate of migration based on the concentration of therapeutic agent dissolved in the matrix, the surface area of the drug core can also be controlled to attain the desired rate of drug migration from the core to the target site. For example, a larger exposed surface area of the core will increase the rate of migration of the treatment agent from the drug core to the target site, and a smaller exposed surface area of the drug core will decrease the rate of migration of the therapeutic agent from the drug core to the target site. The exposed surface area of the drug core can be increased in any number of ways, for example by making the exposed surface tortuous or porous, thereby increasing the surface area available to the core.

The sheath body comprises appropriate shapes and materials to control migration of the therapeutic agent from the drug core. The sheath body houses the core and can fit snugly against the core. The sheath body is made from a material that is substantially impermeable to the therapeutic agent so that the rate of migration of the therapeutic agent may be largely controlled by the exposed surface area of the drug core that is not covered by the sheath body. Typically, migration of the therapeutic agent through the sheath body will be about one tenth of the migration of the therapeutic agent through the exposed surface of the drug core, or less, often being one hundredth or less. In other words, the migration of the therapeutic agent through the sheath body is at least about an order of magnitude less that the migration of the therapeutic agent through the exposed surface of the drug core. Suitable sheath body materials include polyimide, polyethylene terephthalate” (hereinafter “PET”). The sheath body has a thickness, as defined from the sheath surface adjacent the core to the opposing sheath surface away from the core, from about 0.00025″ to about 0.0015″. The total diameter of the sheath that extends across the core ranges from about 0.2 mm to about 1.2 mm. The core may be formed by dip coating the core in the sheath material. Alternatively, the sheath body can be a tube and the core introduced into the sheath as a liquid or slid into the sheath body tube.

The sheath body can be provided with additional features to facilitate clinical use of the implant. For example, the sheath may replaceable receive a drug core that is exchangeable while the retention element and sheath body remain implanted in the patient. The sheath body is often rigidly attached to the retention element as described above, and the core is exchangeable while the retention element retains the sheath body. For example, the sheath body can be provided with external protrusions that apply force to the sheath body when squeezed and eject the core from the sheath body. Another drug core can then be positioned in the sheath body.

The retention element comprises an appropriate material that is sized and shaped so that the implant can be easily positioned in the desired tissue location, for example the punctum or canaliculus. The retention element is mechanically deployable and typically expands to a desired cross sectional shape, for example with the retention element comprising a superelastic shape memory alloy such as Nitinol™. Other materials in addition to Nitinol™ can be used, for example resilient metals or polymers, plastically deformable metals or polymers, shape memory polymers and the like for example spring stainless steel, Eligloy®, tantalum, titanium, cobalt chromium to provide the desired expansion. The retention element may be bio-degradable or non-biodegradable depending on the desired treatment time and whether the patient requires physician follow up. This expansion capability permits the implant to fit in hollow tissue structures of varying sizes, for example canaliculae ranging from 0.3 mm to 1.2 mm (i.e. one size fits all). Although a single retention element can be made to fit canaliculae from 0.3 to 1.2 mm across, a plurality of alternatively selectable retention elements can be used to fit this range if desired, for example a first retention element for canaliculae from 0.3 to 0.9 mm and a second retention element for canaliculae from 0.9 to 1.2 mm. The retention element has a length appropriate to the anatomical structure to which the retention element attaches, for example a length of about 3 mm or less for a retention element positioned near the punctum of the canaliculus.

Although the sheath body and drug core are attached to one end of the retention element as described above, in many embodiments the other end of retention element is not attached to drug core and sheath body so that the retention element can slide over the sheath body and drug core while the retention element expands. This sliding capability on one end is desirable as the retention element will typically shrink in length as the retention element expands in width to assume the desired cross sectional width. In addition, the core of the device may be replaceable with the sheath body remaining in place. Alternatively, the sheath body may be replaceable within the retention element to provide for exchange of a the drug core to replenish the supply of therapeutic agent to the device.

The occlusive element comprises an appropriate material that is sized and shaped so that the implant can at least partially inhibit, even block, the flow of fluid through the hollow tissue structure, for example lacrimal fluid through the canaliculus. The occlusive material shown is a thin walled membrane of a biocompatible material, for example silicone, that can expand and contract with the retention element. The occlusive element is formed as a separate thin tube of material that is slid over the end of the retention element and anchored to one end of the retention element as described above. Alternatively, the occlusive element can be formed by dip coating the retention element in a biocompatible polymer, for example silicone polymer. The thickness of the occlusive element can be in a range from about 0.03 mm to about 0.15 mm, and often from about 0.05 mm to 0.1 mm.

FIG. 1C shows a perspective view of a sustained release implant 102 with a coil retention element 132, according to an embodiment of the present invention. Retention element 132 comprises a coil and retains a drug core 112. Drug core 112 is partially covered. The sheath body comprises a first component 122A that covers a first end of drug core 112 and a second component 122B that covers a second end of the drug core. An occlusive element can be placed over the retention element or the retention element can be dip coated as described above.

FIG. 1D shows a perspective view of a sustained release implant 104 with a retention element 134 comprising struts, according to an embodiment of the present invention. Retention element 134 comprises longitudinal struts and retains a drug core 114. Drug core 114 is covered with a sheath body 124 over most of drug core 114. The drug core releases therapeutic agent through an exposed end and sheath body 124 is annular over most of the drug core as described above. An occlusive element can be placed over the retention element or the retention element can be dip coated as described above.

FIG. 1E shows a perspective view of a sustained release implant 106 with a cage retention element 136, according to an embodiment of the present invention. Retention element 136 comprises several connected strands of metal (such as a mesh or lattice, or helical structure) and retains a drug core 116. Drug core 116 is covered with a sheath body 126 over most of drug core 116. The drug core releases therapeutic agent through an exposed end and sheath body 126 is annular over most of the drug core as described above. An occlusive element can be placed over the retention element or the retention element can be dip coated as described above.

FIG. 1F shows a perspective view of a sustained release implant comprising a core and sheath, according to an embodiment of the present invention. Drug core 118 is covered with a sheath body 128 over most of drug core 118. The drug core releases therapeutic agent through an exposed end and sheath body 128 is annular over most of the drug core as described above. The rate of therapeutic agent release is controlled by the surface area of the exposed drug core and materials comprised within drug core 118. Such an implant can be implanted in ocular tissues, for example below conjunctival tissue layer 9 of the eye and either above sclera tissue layer 8, as shown in FIG. 1F, or only partially within the scleral tissue layer so as not to penetrate the scleral tissue. It should be noted that drug core 118 can be used with any of the retention elements and occlusive elements as described herein. In an embodiment, the drug core is implanted between sclera 8 and conjunctiva 9 without sheath body 128. In this embodiment without the sheath body, the physical characteristics of the drug core can be adjusted to compensate for the increased exposed surface of drug core, for example by reducing the concentration of dissolved atropine in the drug core matrix as described herein.

The cores and sheath bodies described herein can be implanted in a variety of tissues in several ways. Many of the cores and sheaths described herein, in particular the structures described with reference to FIGS. 2A to 2J can be implanted alone as punctal plugs. Alternatively, many of the cores and sheath bodies described herein can comprise a drug core, sheath body, and/or the like so as to be implanted with the retention elements and occlusive elements described herein.

FIG. 2A shows a cross sectional view of a sustained release implant 200 with core comprising an enlarged exposed surface area, according to an embodiment of the present invention. A drug core 210 is covered with a sheath body 220. Sheath body 220 includes an opening 220A. Opening 220 has a diameter that approximates the maximum cross sectional diameter of drug core 210. Drug core 210 includes an exposed surface 210E, also referred to as an active surface. Exposed surface 210E includes 3 surfaces: an annular surface 210A, a cylindrical surface 210B and an end surface 210C. Annular surface 210A has an outer diameter that approximates the maximum cross sectional diameter of core 210 and an inner diameter that approximates the outer diameter of cylindrical surface 210B. End surface 210C has a diameter that matches the diameter of cylindrical surface 210B. The surface area of exposed surface 210E is the sum of the areas of annular surface 210A, cylindrical surface 210B and end surface 210C. The surface area may be increased by the size of cylindrical surface area 210B that extends longitudinally along an axis of core 210.

FIG. 2B shows a cross sectional view of a sustained release implant 202 with a core 212 comprising an enlarged exposed surface area 212A, according to an embodiment of the present invention. A sheath body 222 extends over core 212. The treatment agent can be released from the core as described above. Exposed surface area 212A is approximately conical, can be ellipsoidal or spherical, and extends outward from the sheath body to increase the exposed surface area of drug core 212.

FIGS. 2C and 2D show perspective and cross sectional views, respectively, of a sustained release implant 204 with a drug core 214 comprising a reduced exposed surface area 214A, according to an embodiment of the present invention. Drug core 214 is enclosed within a sheath body 224. Sheath body 22 includes an annular end portion 224A that defines an opening through which drug core 214 extends. Drug core 214 includes an exposed surface 214A that releases the therapeutic agent. Exposed surface 214A has a diameter 214D that is less than a maximum dimension, for example a maximum diameter, across drug core 214.

FIG. 2E shows a cross sectional view of a sustained release implant 206 with a drug core 216 comprising an enlarged exposed surface area 216A with castellation extending therefrom, according to an embodiment of the present invention. Drug core 216 includes an indentation 2161. The castellation includes several fingers 216F extending from the indentation. Core 216 is covered with a sheath body 226. Sheath body 226 is open on one end to provide an exposed surface 216A on drug core 216. Indentation 2161 has the shape of an inverted cone. Several fingers 216F extend outward from indentation 2161 to provide an increase in surface area of exposed surface 216A. Sheath body 226 also includes fingers and has a castellation pattern that matches core 216.

FIG. 2F shows a perspective view of a sustained release implant 250 comprising a core with folds, according to an embodiment of the present invention. Implant 250 includes a core 260 and a sheath body 270. Core 260 has an exposed surface 260A on the end of the core that permits drug migration to the surrounding tear or tear film fluid. Core 260 also includes folds 260F. Folds 260F increase the surface area of core that contains the drug to be delivered within the volume of the implant. With this increase in exposed surface area, folds 260F increase migration of the therapeutic agent from core 260 into the tear or tear film fluid and target treatment area. Folds 260F are formed so that a channel 260C is formed in core 260. Channel 260C connects to the end of the core to an opening in exposed surface 260A and provides for the migration of treatment agent. Thus, the total exposed surface area of core 260 includes exposed surface 260A that is directly exposed to the tear or tear film fluid and the surfaces of folds 260F that are exposed to the tear or tear film fluids via connection of channel 260C with exposed surface 260A and the tear or tear film fluid.

FIG. 2G shows a perspective view of a sustained release implant with a core comprising a channel with a series of protrusions and/or cavities extending from the central axis, according to an embodiment of the present invention. Implant 252 includes a core 262 and sheath body 272. Core 262 has an exposed surface 262A on the end of the core that permits drug migration to the surrounding tear or tear film fluid. Core 262 also includes a channel 262C. Channel 262C increases the surface area of the channel with a porous internal surface 262P formed on the inside of the channel against the core. Channel 262C extends to the end of the core near exposed surface 262A of the core. The surface area of core that is exposed to the surrounding fluid tear or tear film fluid can include the inside of core 262 that is exposed to channel 262C. This increase in exposed surface area can increase migration of the therapeutic agent from core 262 into the tear or tear film fluid and target treatment area. Thus, the total exposed surface area of core 262 can include exposed surface 260A that is directly exposed to the tear or tear film fluid and porous internal surface 262P that is exposed to the tear or tear film fluids via connection of channel 262C with exposed surface 262A and the tear or tear film fluid.

FIG. 2H shows a perspective view of a sustained release implant 254 with a core 264 comprising porous channels to increase drug migration, according to an embodiment of the invention. Implant 254 includes core 264 and sheath body 274. Exposed surface 264A is located on the end of core 264, although the exposed surface can be positioned at other locations. Exposed surface 264A permits drug migration to the surrounding tear or tear film fluid. Core 264 also includes porous channels 264C. Porous channels 264C extend to exposed surface 264. Porous channels 264C are large enough that tear or tear film fluid can enter the porous channels and therefore increase the surface area of core 264 that is in contact with tear or tear film fluid. The surface area of the core that is exposed to the surrounding fluid tear or tear film fluid includes the inner surfaces of channels 264C. With this increase in exposed surface area, porous channels 264C increase migration of the therapeutic agent from core 264 into the tear or tear film fluid and target treatment area. Thus, the total exposed surface area of core 264 includes exposed surface 264A that is directly exposed to the tear or tear film fluid and internal surface that is exposed to the tear or tear film fluids via connection of porous channel 262C with exposed surface 264A and the tear or tear film fluid.

FIG. 2I shows a perspective view of a sustained release implant 256 with a drug core 266 comprising a convex exposed surface 266A, according to an embodiment of the present invention. Drug core 266 is partially covered with a sheath body 276 that extends at least partially over drug core 266 to define convex exposed surface 266A. Sheath body 276 comprises a shaft portion 276S. Convex exposed surface 266A provides an increased exposed surface area above the sheath body. A cross sectional area of convex exposed surface 266A is larger than a cross sectional area of shaft portion 276S of sheath body 276. In addition to the larger cross sectional area, convex exposed surface 266A has a larger surface area due to the convex shape which extends outward from the core. Sheath body 276 comprises several fingers 276F that support drug core 266 in the sheath body and provide support to the drug core to hold drug core 266 in place in sheath body 276. Fingers 276F are spaced apart to permit drug migration from the core to the tear or tear film fluid between the fingers. Protrusions 276P extend outward on sheath body 276. Protrusions 276P can be pressed inward to eject drug core 266 from sheath body 276. Drug core 266 can be replaced with another drug core after an appropriate time, for example after drug core 266 has released most of the therapeutic agent.

FIG. 2J shows a side view of a sustained release implant 258 with a core 268 comprising an exposed surface area with several soft brush-like members 268F, according to an embodiment of the present invention. Drug core 268 is partially covered with a sheath body 278 that extends at least partially over drug core 268 to define exposed surface 268A. Sheath body 278 comprises a shaft portion 278S. Soft brush-like members 268F extend outward from drug core 268 and provide an increased exposed surface area to drug core 268. Soft brush-like members 268F are also soft and resilient and easily deflected such that these members do not cause irritation to neighboring tissue. Although drug core 268 can be made of many materials as explained above, silicon is a suitable material for the manufacture of drug core 268 comprises soft brush like members 268F. Exposed surface 268A of drug core 268 also includes an indentation 2681 such that at least a portion of exposed surface 268A is concave.

FIG. 2K shows a side view of a sustained release implant 259 with a drug core 269 comprising a convex exposed surface 269A, according to an embodiment of the present invention. Drug core 269 is partially covered with a sheath body 279 that extends at least partially over drug core 269 to define convex exposed surface 269A. Sheath body 279 comprises a shaft portion 279S. Convex exposed surface 269 provides an increased exposed surface area above the sheath body. A cross sectional area of convex exposed surface 269A is larger than a cross sectional area of shaft portion 279S of sheath body 279. In addition to the larger cross sectional area, convex exposed surface 269A has a larger surface area due to the convex shape that extends outward on the core. A retention element 289 comprising a coil of wire is attached to sheath body 279. Retention element 289 can be dip coated to make retention element 289 biocompatible.

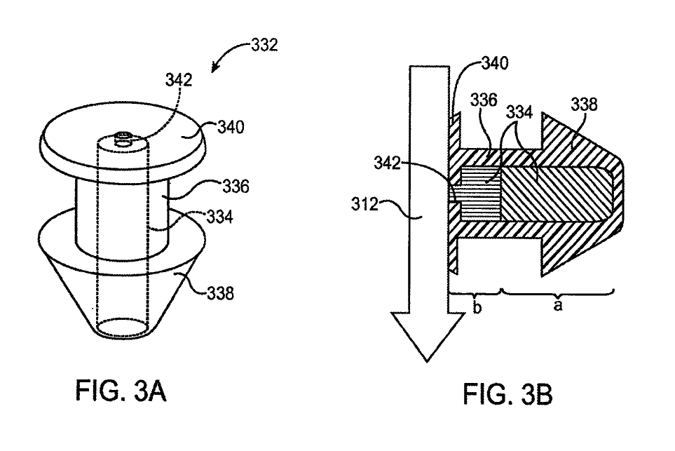

FIGS. 3A to 3C show retention elements that encompass punctual plugs and structures to release therapeutic agents that encompass reservoirs, according to embodiments of the present invention. Structures suitable for incorporation with the present invention are described in U.S. Pat. No. 6,196,993, entitled “Ophthalmic insert and method for sustained release of medication to the eye”, issued in the name of Cohan on Mar. 6, 2001, the full disclosure of which is incorporated herein by reference. The reservoir can include any of the therapeutic agents described herein to treat optical defects of the eye, for example atropine to treat myopia of the eye. The migration of the drug from the reservoir may occur by diffusion, although other migration mechanisms are possible.

FIG. 3A shows a perspective view of a punctual plug with a reservoir, according to an embodiment of the present invention. An ophthalmic insert 332 is shown in the form of a punctal occluder with a reservoir 334 designed to store and release therapeutic agent onto the surface of the eye in a continuous, long-term manner. Ophthalmic insert 332 can be molded or otherwise formed from a flexible material, such as silicone, that is impermeable to the therapeutic agent, which will fill the reservoir 334. Reservoir 334 is formed by a channel through the interior of a body portion 336 of insert 332. Preferably, body portion 336 is flexible, and may even be accordion-shaped to provide the capability of lengthwise expansion as it is filled with the therapeutic agent.

Still referring to FIG. 3A, a collarette 340 anchors the insert 332 to the exterior of the lacrimal punctum and is provided with a pore 342 in fluid communication with reservoir 334. In order to control the delivery of a specific therapeutic agent, the geometry of pore 342 may be customized as explained in U.S. Pat. No. 6,196,993, previously incorporated herein by reference. Through pore 342, therapeutic agent is deployed from reservoir 334 into the tears of the lacrimal lake where the therapeutic agent mixes, as eye drops do, with the tears and penetrates the eye to have the intended pharmacological effect. Although not required, an enlarged bulb portion 238 may be provided to help secure the insert 332 within the canaliculus and also to provide additional volume for reservoir 334 as shown.

FIG. 3B shows a schematic representation of a preferred configuration of medication within the reservoir and its contact with the external tear flow, according to an embodiment of the present invention. The reservoir 334 includes a region (a) containing the most concentrated form of the medication, in either a solid or liquid state. The medication diffuses from region (a) into an adjacent region (b), nearest the pore 342, comprising a saturated solution of the medication. The rate-controlling pore 342 can be formed with desired dimensions at the time the insert 332 is made, or pore 342 could be sized appropriately by retrofitting insert 332 with an apertured cap of appropriate geometry fit over reservoir 334. In an alternative embodiment, pore 342 could be provided in the form of an imperforate material placed over the collarette 340 that is permeable to the passage of the medication.

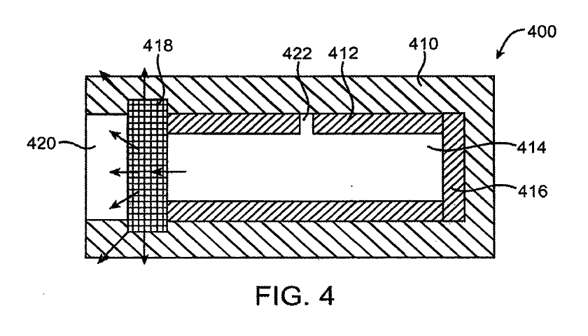

FIG. 4 shows a retention element that encompass a tube, for example a tube used to form a punctual plug, and a structure to release therapeutic agents that encompass a drug reservoir at least partially enclosed with a permeable layer, according to an embodiment of the present invention. Structures suitable for incorporation with the present invention are described in U.S. Pat. App. Pub. No. 2004/0208910, entitled “Sustained release device and method for ocular delivery of adrenergic agents”, published in the name of Ashton on Oct. 21, 2004, the full disclosure of which is incorporated herein by reference. The reservoir can include any of the therapeutic agents described herein to treat optical defects of the eye, for example atropine to treat myopia of the eye. The retention element comprises any of the structures described in the '910 publication used to retain the drug reservoir at the intended location near the eye.

FIG. 4 schematically illustrates an enlarged cross-sectional illustration of a sustained release drug delivery device with a reservoir and a permeable plug. A device 300 includes a permeable outer layer 310, a substantially impermeable inner tube 312, a reservoir 314, a substantially impermeable cap 316, and a permeable plug 318. A port 320 communicates plug 318 with the exterior of the device, as described above with respect to port 224 and plug 216. Inner tube 312 and cap 316 can be formed separately and assembled together, or the inner tube and the cap can be formed as a single, integral, monolithic element. The provision of permeable outer layer 310 allows the therapeutic agent(s) in reservoir or drug core 314 to flow through the outer layer in addition to port 320, and thus assists in raising the overall delivery rate. The material out of which outer layer 310 is formed can be specifically chosen for its ability to adhere to the underlying structures, cap 316, tube 312, and plug 318, and to hold the entire structure together. Optionally, a hole or holes 322 can be provided through inner tube 312 to increase the flow rate of the therapeutic agent(s) from reservoir 314.

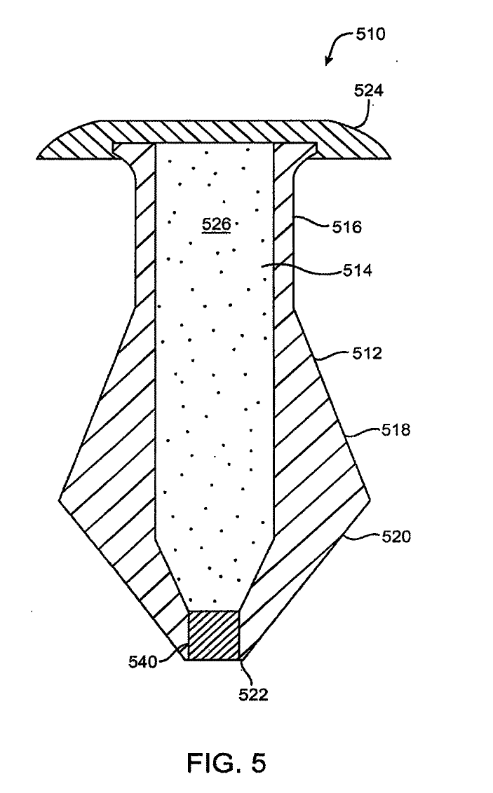

FIG. 5 shows a retention elements that encompasses a punctual plug, and a structure to release therapeutic agents that encompasses a drug reservoir enclosed with a material permeable to the drug, according to an embodiment of the present invention. Structures suitable for incorporation with the present invention are described in U.S. Pat. App. Pub. Nos. 2004/0020253, entitled “Implantable device having controlled release of medication and method of manufacturing the same”, published in the name of Prescott on Jan. 26, 2006; and U.S. App. Pub. No. 2006/0020248, entitled “Lacrimal insert having reservoir with controlled release of medication and method of manufacturing the same”, published in the name of Prescott on Jan. 26, 2006, the full disclosures of which are incorporated herein by reference. The reservoir can include any of the therapeutic agents described herein to treat optical defects of the eye, for example medications to treat optical defects of the eye.

FIG. 5 schematically illustrates a lacrimal insert in the shape of a punctum plug 510 for insertion into a lacrimal puncta. The punctum plug 510 includes a body 512 defining a reservoir 514, a neck portion 516, a flared portion 518, and a tapered portion 520 terminating in a tip 522. A non-porous head 524 is provided over the neck portion 516 of the body 512, and these enclose the reservoir. A medication 526 is provided in the reservoir. In accord with one aspect of the invention, the body 512 and head 524 are made of different materials, with the body 512 being made from a biocompatible, preferably soft and flexible first material which is relatively impermeable to the medication, and the head 524 being made from a biocompatible, preferably soft and flexible second material which is permeable to the medication.

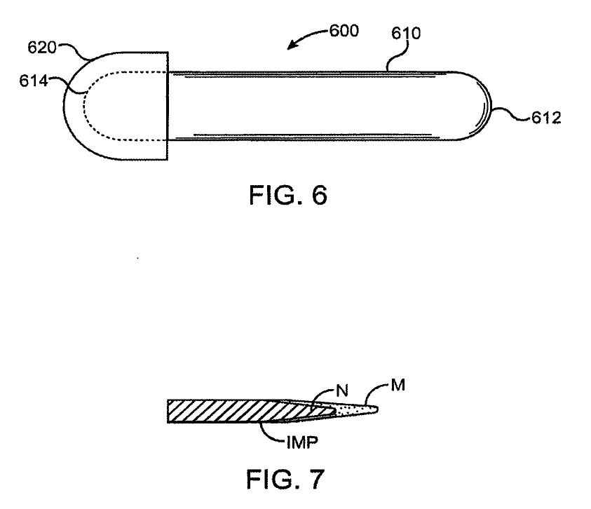

FIG. 6 shows a punctual plug having materials to release therapeutic agents (e.g. coatings and/or biodegradable polymers) according to embodiments of the present invention. Structures suitable for use with the present invention are described in PCT/US2005/023848 published as International Pub. No. WO 2006/014434, entitled “TREATMENT MEDIUM DELIVERY DEVICE AND METHODS FOR DELIVERY OF SUCH TREATMENT MEDIUMS TO THE EYE USING SUCH A DELIVERY DEVICE”, in the name of Lazar on Feb. 9, 2006. The biodegradable polymer can include any of the therapeutic agents described herein to treat optical defects of the eye, for example a treatment medium such as atropine to treat myopia of the eye.

FIG. 6 shows a treatment medium delivery device 600 according an embodiment of the present invention. The treatment medium delivery device 600 includes a first body portion 610 and a second body portion 620. The second body portion 620 is generally configured and arranged so as to include the therapeutic agent or treatment medium that is to be dispensed.

The first body portion 610 is sized, configured and arranged so as to be removably inserted and secured in an opening provided in the eye, more particularly, a portion of the body proximal the eye. More particularly, the first body portion 610 is sized, configured and arranged such that when the first body portion is inserted into the opening it is secured within the opening so it does not fall or come out as a result of normal and expected bodily function, such as for example, blinking of the eyelids and any laxity of the eye. In particular exemplary embodiments, the opening in the eye is a punctum of the eye for a mammalian body that is fluidly coupled to a lacrimal canaliculus, and the treatment medium delivery device is configured and arranged so it remains secured within the punctum and a portion of the lacrimal canaliculus during normal eye function.

The first body portion 610 is configurable in any number of ways, for example as a solid member, a member having a lumen or passage defined therein, a member having a passage passing through a portion of the first body portion, an open compartment located within the first body portion, and a body structure that corresponds to the structure of a stent. A stent provides a scaffold like structure that can be arranged to form a generally cylindrical shape or a shape that conforms to the opening and passage into which the stent is being inserted. The first body portion 610 also is constructed of any of a number of biocompatible materials as is known to those skilled in the art, including metals such as stainless steel and nitinol (nickel-titanium) and plastics that have strength and material characteristics suitable for the intended use. Such materials of the first body portion 610 also preferably are characterized as being non-toxic and non-sensitizing.

In more particular embodiments, the first body portion includes an end 612 that is configured to facilitate insertion of the first body portion 610 into the opening as well as to minimize significant trauma and/or injury to the tissue of the opening as the first body portion is being inserted therein, hi specific exemplary embodiments, the first body portion end 612 is arcuate and/or generally hemispherical. The first body portion end 612 can be configured so it presents an end that is appropriate for the intended function and use. For example, the end 612 is configurable so as to have a piercing capability if the function and use of the first end portion 610 would involve piercing of tissue or a membrane as the first portion end is being inserted into the body opening.

In an embodiment of the present invention, the second body portion 620 comprises a member, device (e.g., an eluting device, a sustained released device, an encapsulation device) or coating that is applied, secured, attached or bonded to the first body portion second end 614 using any of a number of techniques known to those skilled in the art such as adhesives. Such a second body portion 620 is constituted so as to carry one or more treatment mediums, and provide a delivery vehicle or structure, such as a matrix or medium, that is constituted so it releasably retains the one or more treatment mediums therein so the medium can be released there from under predetermined conditions. Such releasably retaining includes but is to limited to encapsulation of the treatment medium(s) within the structure comprising the delivery vehicle or structure. It also is contemplated that the second body portion 620 can comprise a medium or material, for example a polymer, that is formed, cured or otherwise appropriately processed such that it is bonded to the first body portion second end 614, as a result of such forming, curing, polymerizing or processing. Additional description of the second body portion are described in WO 2006/014434.

FIG. 7 shows a retention element that comprises an elongate member for complete insertion into the canaliculus of the eye and a structure to release therapeutic agents that encompasses a coating on the retention element, according to an embodiment of the present invention. Structures suitable for incorporation with the present invention are described in U.S. Pat. No. 5,053,030, entitled “Intracanalicular implant for horizontal canalicular blockade treatment of the eye”, issued in the name of Herrick on Oct. 1, 1991, the full disclosure of which is incorporated herein by reference. The of therapeutic agent can include a medication, for example a treatment medium such as atropine to treat myopia of the eye.

FIG. 7 shows an implant for complete insertion into the canaliculus of the human eye with medication, according to an embodiment of the present invention. An implant Imp is constructed of two parts, with the second part M having a preselected configuration to be mounted to the nose N of the implant Imp and for loading it with medication. The illustrated configuration for the part M has one end defined to be complementary to the nose end of the part Imp to be carried thereby and a blunt nose for the opposite end. These medications can be loaded onto the intracanalicular implant Imp for timed release dosages to the eye. This release would work as a result of the reflex action of the eye and could be used, for example, to distribute atropine to the muscle of the eye.

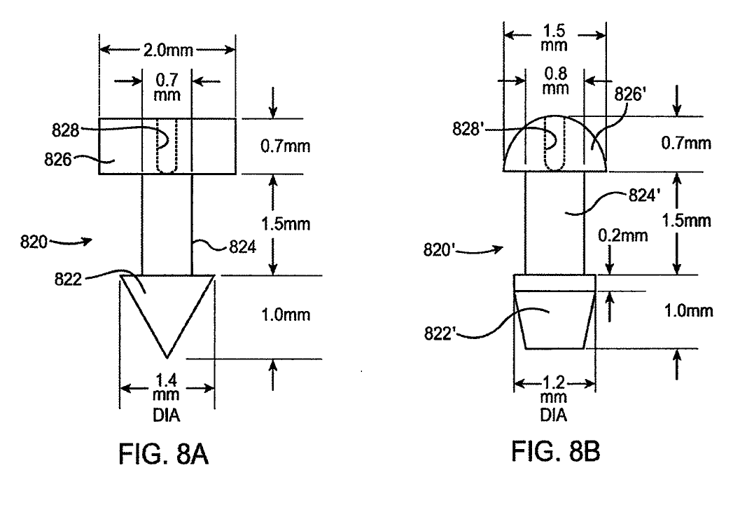

FIGS. 8A and 8B show retention elements that encompass punctual plugs and structures to release therapeutic agents that encompass the head portion of the punctual plug, according to an embodiment of the present invention. Structures suitable for incorporation with the present invention are described in U.S. Pat. No. 3,949,750, entitled “Punctum plug and method for treating keratoconjunctivitis sicca and other ophthalmic aliments using same”, issued in the name of Freeman on Apr. 13, 1976, the full disclosure of which is incorporated herein by reference. The head portion can include any of the therapeutic agents described herein to treat optical defects of the eye, for example atropine to treat myopia of the eye.

In the treatment of ophthalmic ailments where it is desired to prevent or decrease the drainage of lacrimal fluid and/or medication from the eye, the punctal aperture in either or both of the upper and lower lids are to be blocked by a removable plug member 820, two respective embodiments of which are shown in FIGS. 8A and 8B. Referring initially to the embodiment of FIG. 8A, the punctum plug 820 has a projecting tip or barb portion 822, a middle neck or waist portion 824 of somewhat smaller diameter than the tip, and a smooth disc-like head portion 826 of relatively larger diameter. The plug embodiment 820′ of FIG. 8B is of generally similar dimensions to the first-described embodiment with a somewhat blunted tip or barb portion 822′, a cylindrical middle portion 824′ of substantially the same dimension, and a dome-shaped head portion 826′ of somewhat smaller diameter than its counterpart in the embodiment of FIG. 8A. The head portion 826, 826′ of both embodiments may be provided, if desired as an alternative to grasping it with forceps, with a central bore opening 828, 828′ adapted to receive the projecting tip of an inserter tool to provide a releasable grip on the plug as it is manipulated for insertion, as hereinafter described.

In certain embodiments of the invention the plugs 820, 820′, particularly the head portion 828, 828′, may be of medication-impregnable porous material such as HEMA hydrophilic polymer, or may be otherwise adapted as with capillaries or the like, to store and slowly dispense ophthalmic drugs to the eye as they are leached out by the lacrimal fluids.

In an embodiment, therapeutic agents as described herein are incorporated into a punctual plug as described in U.S. App. Pub. No. 2005/0197614, the full disclosure of which is incorporated herein by reference. A gel can be used to form a punctual plug, and the gel can swell from a first diameter to a second diameter in which the second diameter is about 50% greater than the first diameter. The gel can be used to entrap active therapeutic agents, for example within a microporous structure in which the agent is uniformly dispersed, and the gel can slowly elute therapeutic agents into the patient. Various therapeutic agents are described in U.S. Provisional Application No. 60/550,132, entitled “Punctum Plugs, Materials, And Devices”, the full disclosure of which is incorporated herein by reference, and may be combined with the gels and devices described herein.

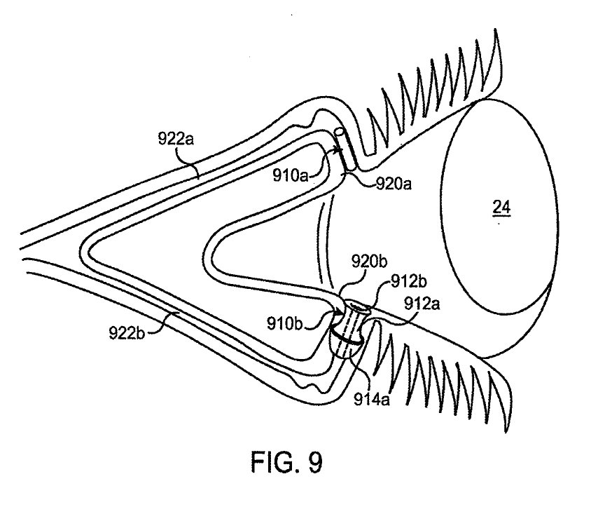

FIG. 9 shows a retention element that encompasses a punctual plug and a retention element that encompasses a hollow implant, and structures to release therapeutic agents that encompass coatings applied to the retention elements, according to an embodiment of the present invention. Structures suitable for incorporation with the present invention are described in U.S. Pat. App. Pub. No. 2005/0232972, entitled “Drug delivery via punctual plug”, published in the name of Odrich on Oct. 20, 2005, the full disclosure of which is incorporated herein by reference.

FIG. 9 shows a punctal plug generally designated 910, having a stem 912 for insertion into the punctal aperture 920 of an eye 924, and along the canaliculus 922 communicating with the aperture. Plug 910 has a large stopper structure 914 connected to the outer end of stem 912 for seating against the aperture 920 and sealing the canaliculus 922 against the flow of tears onto the surface of an eye 924. The same or similar numerals are used to designate functionally similar parts, for example upper and lower canaliculi 922a and 922b, each with implants 910a and 910b, respectively. Implant 910a is a substantially cylindrical and solid collagen plug that has been inserted into the upper punctum or tear duct 920a, to block the flow of tears while lower implant 910b is hollow like a straw for the passage of tears. Implant 910b includes a tapered shaft or stem 912a with a flared open end 912b immobilized at the lower punctum 920b. A mushroom shaped inner stopper 914a is formed at the opposite end of shaft 912a for further setting the location of the implant in the tear duct. The implants shown can be used in any desired combination, for example implant 910a can be positioned in the lower canaliculus and implant 910b can be positioned in the upper canaliculus. Alternatively, each type of implant (e.g. 910b) can be positioned in both canaliculi.

The active agent, e.g. a medicine or medication is applied, e.g. in one or more bands of polymer material at the inner end of the stem, or on the outer end of the stopper, or over some or all of the surfaces of the implants of FIG. 9, or otherwise. Polymer that is absorbent to the agent is preferable so that sufficient agent is present and available for discharge into the surrounding tissues. A porous or absorbent material can alternatively be used to make up the entire plug or implant which can be saturated with the active agent.

Unlike the tear stopping punctal plug, the hollow implant provides a very different drug administering method, scheme and structure. The hollow implant 910b of FIG. 9 is particularly useful in that the active agent can be applied to, or is otherwise available at the inner surface or interior of the implant, and is uniquely structured to pass tears and thus administer the active agent to the tear stream in a fashion that is controlled by the flow of tears which thus act as the carrier for the agent.

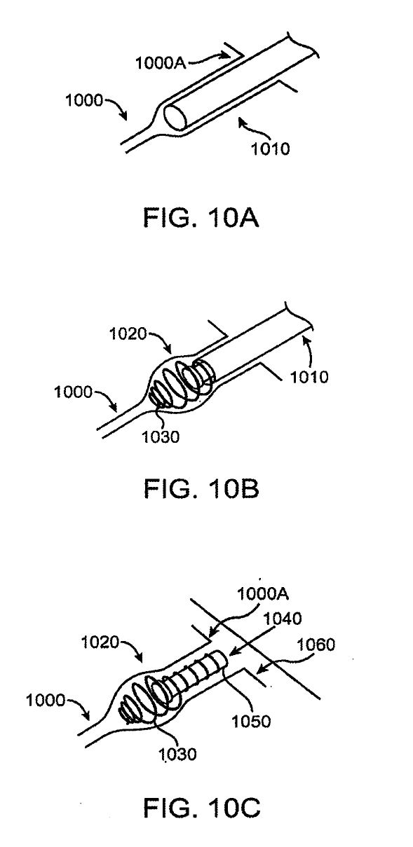

FIGS. 10A to 10C show deployment of a sustained release implant, according to an embodiment of the present invention. As shown in FIG. 10A, a deployment instrument 1010 is inserted into a canaliculus 1000 through a punctum 1000A. A sustained release implant 1020 is loaded into a tip of deployment instrument 1010. As shown in FIG. 10B, an outer sheath of deployment instrument 1010 is withdrawn to expose a retention element 1030 of sustained release implant 1020. As shown in FIG. 10C, deployment instrument 1010 has been removed and sustained release implant 1020 is implanted in canaliculus 1000. A drug core 1040 is attached retention element 1030 and retained in the canaliculus. An outer body sheath 1050 covers at least a portion of drug core 1040 and drug core 1040 releases a therapeutic agent into a liquid tear or tear film 1060 near punctum 1000A of canaliculus 1000.

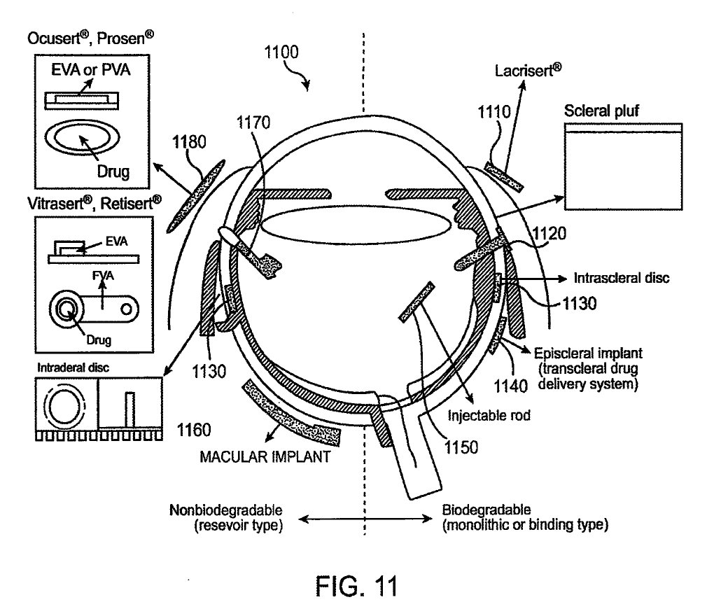

FIG. 11 shows sustained release therapeutic agent implants and implant locations on or near an eye 1100, according to embodiments of the present invention. The sustained release implant can comprises many of the structures used with Lacrisert®, scleral plugs, intrascleral discs, episcleral implants, injectable rods, macular implants, intrascleral discs, Vitrasert®, Retisert®, Ocusert® and/or Prosert® implants. Similar structures are shown in the publication by Yasukawa, et al., “Expert Opinion on Drug Delivery”, Volume 3, Number 2, 1 Mar. 2006, pp. 261-273(13), Published by Informa Healthcare. A sustained release implant 1110 may comprise many structures of Lacrisert™ implants for administration into the inferior cul-de-sac of the eye, which are available from Merck & CO., Inc. of Whitehouse Station, N.J. A sustained release implant 1120 may comprise many structures of a scleral plug implant for administration into the sclera and/or vitreous humor of the eye. A scleral plug and/or tack is described in U.S. Pat. No. 5,466,233, the full disclosure of which is incorporated herein by reference. A sustained release implant 1130 may comprise many structures of a scleral disc implant for administration into the sclera. An intrascleral disc can be inserted into the sclera tissue layer. A sustained release implant 1140 may comprise many structures of an episcleral disc implant that can be placed near a surface of the sclera and provide a trans-scleral drug delivery system. A sustained release implant 1150 may comprise many structures of a injectable rod for injection into the aqueous humor, the sclera and or lacrimal ducts. A sustained release implant 1160 may comprise many structures of a macular implant for implantation near a macular tissue of the eye. A sustained release implant 1170 may comprise many structures of Vitrasert® and/or Retisert® implants. Vitrasert® and Retisert® implants are commercially available from Chiron Ophthalmics, a subsidiary Bausch and Lomb of Rochester, N.Y. Ocusert® implants are commercially available from Alza, a subsidiary of Johnson & Johnson of New Brunswick, N.J. Prosert® implants are commercially available from Novartis of Basel, Switzerland.

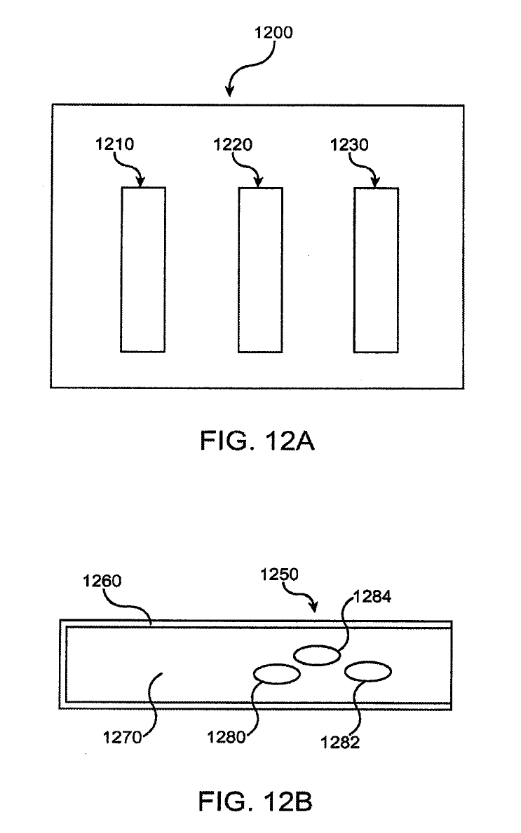

FIG. 12A shows a device 1200 for treating optical defects of the eye that comprises a sustained release implant that releases a therapeutic agent to treat the optical defect of the eye and additional sustained release implants to counteract side effects of the therapeutic agent. Device 1200 comprises a sustained release implant 1210 that releases a therapeutic agent as described above. Device 1200 comprises a sustained release implant 1220 that releases a counteractive agent that counteracts a first side effect of the therapeutic agent. As the therapeutic agent may have more than one side effect, device 1200 may comprises a sustained release implant 1230 that counteracts a second side effect of the therapeutic agent. The sustained release implants may be simultaneously located in many of the locations of or near the eye as described above. In a preferred embodiment, sustained release implant 1210 may release atropine. One side effect of atropine is pupil dilation that can be associated with photophobia. Sustained release implant 1220 may release a miotic drug as a counteractive agent to counteract the dilation of the pupil caused by the therapeutic agent. Another possible side effect of atropine is glaucoma, and sustained release implant 1230 may release an anti-glaucoma drug as a counteractive agent to avoid glaucoma.

FIG. 12B shows a sustained release implant 1250 that releases a therapeutic agent to treat an optical defect of the eye and releases counteractive agents that counteract side effects of the therapeutic agent, according to embodiments of the present invention. Sustained release implant 1250 may comprise a sheath body 1260 and a drug core 1270. Sustained release implant 1250 may be placed in many of the locations of or near the eye as described above. Drug core 1270 comprises a therapeutic agent 1280 to treat an optical defect of the eye. Drug core 1270 may comprise a counteractive agent 1282 to counteract a side effect of therapeutic agent 1280. In a preferred embodiment, sustained release implant 1250 may release atropine. Therapeutic agent 1282 may comprise a miotic drug as a counteractive agent to counteract the dilation of the pupil caused by the therapeutic agent. Another possible side effect of atropine is glaucoma, and therapeutic agent 1284 may release an anti-glaucoma drug as a counteractive agent to avoid glaucoma. The therapeutic agent, the miotic drug and the anti-glaucoma drug may be released together from sustained release implant 1250.

Although the invention has been described by way of the specific embodiments described above, one will recognize various modifications and alterations that can be readily made and that are within the scope and spirit of the invention. Therefore, the present invention is limited only by the following claims and the full scope of their equivalents.

1. An implant for use with an eye, the implant comprising: an at least partially implantable structure; and
a therapeutic agent deliverable from the structure into the eye to therapeutically effect and/or stabilize a refractive property of the eye,
wherein the structure includes at least one surface configured to release a therapeutic quantity of the therapeutic agent into tear or tear film fluid of the eye when exposed thereto.

2-51. (canceled)

52. The implant of claim 1, wherein the refractive property of the eye comprises at least one of myopia, hyperopia, or astigmatism.

53. The implant of claim 1, wherein the therapeutic agent comprises a composition configured to therapeutically effect or stabilize the refractive property of the eye when delivered into at least one of a sclera, a vitreous humor, an aqueous humor, or a ciliary muscle of the eye.

54. The implant of claim 1, wherein the therapeutic agent comprises at least one of a mydriatic or a cycloplegic drug.

55. The implant of claim 54, wherein the cycloplegic drug comprises at least one of atropine, cyclopentolate, succinylcholine, homatropine, scopolamine, or tropicamide.

56. The implant of claim 1, further comprising a retention element attached to the structure to retain the structure along a natural tissue surface of, or adjacent to, the eye.

57. The implant of claim 56, wherein the retention element is shaped to retain the structure in or adjacent at least one of a punctal duct, a scleral tissue, or a conjunctival tissue.

58. The implant of claim 57, wherein the retention element comprises a punctal insert to retain the structure in the punctal duct.

59. The implant of claim 1, wherein the structure comprises at least one of a reservoir, a matrix, a solution, a surface coating, or a bioerodable material.

60. The implant of claim 1, wherein the structure comprises a drug core and a layer disposed at least partially over the drug core to inhibit release of the therapeutic agent through the layer.

61. The implant of claim 60, wherein the layer comprises or forms an opening sized and shaped to release the therapeutic agent therethrough.

62. A therapeutic implant comprising:
a structure;
a punctal insert to retain the structure adjacent to an eye; and
a therapeutic agent deliverable from the structure into the eye to therapeutically effect and/or stabilize one or more refractive properties of the eye.

63. The implant of claim 62, wherein the structure is configured to release a therapeutic quantity of the therapeutic agent throughout a time period of at least one week when the at least one surface is exposed to the tear or tear film fluid.

64. The implant of claim 62, wherein the structure is configured to release a therapeutic quantity of the therapeutic agent over a time period from about one to twelve months after being implanted.

65. The implant of claim 62, wherein the structure comprises one or more particles of the therapeutic agent, the particles independently releasing the therapeutic agent therefrom when the structure is implanted and configured to provide a substantially uniform release rate.

66. The implant of claim 62, further comprising a counteractive agent to avoid a side effect of the therapeutic agent.

67. The implant of claim 66, wherein the counteractive agent comprises at least one of an anti-glaucoma drug or a miotic drug.

68. The implant of claim 67, wherein the anti-glaucoma drug comprises at least one of a sympathomimetic, a parasympathomimetic, a beta blocking agent, a carbonic anhydrase inhibitor, or prostaglandin analogue.

69. The implant of claim 67, wherein the anti-glaucoma drug comprises at least one of Apraclonidine, Brimonidine, Clonidine, Dipivefrine, Epinephrine, Aceclidine, Acetylcholine, Carbachol, Demecarium, Echothiophate, Fluostigmine, Neostigmine, Paraoxon, Physostigmine, Pilocarpine, Acetazolamide, Brinzolamide, Diclofenamide, Dorzolamide, Methazolamide, Befunolol, Betaxolol, Carteolol, Levobunolol, Metipranolol, Timolol, Bimatoprost, Latanoprost, Travoprost, Unoprostone, Dapiprazole, or Guanethidine.

70. A method of treating an optical defect of an eye with a therapeutic agent, the method comprising:
implanting a structure into a tissue of or near the eye; and
releasing a therapeutic agent from the implanted structure into a tear or tear film of the eye to therapeutically effect and/or stabilize a refractive property of the eye.

71. The method of claim 70, wherein releasing the therapeutic agent includes releasing a therapeutic amount of the therapeutic agent over a period of time from about one to twelve months after the structure is implanted into the tissue of or near the eye.

72. The method of claim 71, wherein releasing the therapeutic agent includes continuously releasing a therapeutic amount of the therapeutic agent over the period of time.

73. The method of claim 70, wherein implanting the structure includes at least partially anchoring the structure within or to a punctum.

74. The method of claim 70, comprising releasing a counteractive agent from the implanted structure and/or another structure to counteract a side effect of the therapeutic agent.

75. The method of claim 74, wherein releasing the counteractive agent includes releasing at least one of an anti-glaucoma drug or a miotic drug.
